(19) United States
(12) Patent Application Publication
(10) Pub. No.: US 2011/0308528 A1
(43) Pub. Date: Dec. 22, 2011

(54) Sleep disorder relief device and method

(76) Inventor: Jean-Paul Ciardullo, New York, NY (US)

(21) Appl. No.: 12/803,244

(22) Filed: Jun. 22, 2010

(51) Int. Cl. A61F 5/56

(52) U.S. Cl. 128/848

(57) ABSTRACT

The device has first and second flexible straps forming loops with a soft chin portion fitting under a wearer's chin, and a portion extending around the head, with a frontal bridge portion secured between opposite sides of the loops and being stretched over the front of the wearer's head to pull the lower jaw forwardly and hold it there during sleep. The points of attachment of the bridge easily can be adjusted by the user to maximize effectiveness and comfort.

### FIELD OF THE INVENTION

The present invention relates to devices and methods for relieving sleep disorders. More particularly, the invention relates to devices for and methods of urging the lower jaw of a person forwardly to reduce blockage of a user's breathing passage to alleviate snoring and sleep apnea.

### DISCUSSION OF RELATED ART

It is well known that a person's breathing passage may become partially obstructed if the lower jaw moves to a position that is excessively posterior to its normal resting position. More specifically, when a person falls asleep, the jaw and throat muscles relax, often allowing the lower jaw to fall backwards into a position that may result in obstructed breathing and contribute to snoring and sleep apnea. This problem is exacerbated when gravity pulls the lower jaw back, as when the person is lying in a supine position, that it, on his or her back. The problem is also exacerbated by backwards pressure on the lower jaw upon contact with pillows or bedding.

It is known that the lower jaw's obstruction of the air passage can be alleviated by employing a device to urge the lower jaw forwardly, that is towards the person's face.

One way that has been proposed to do this is to employ a dental device, positioned within the mouth and affixed to the teeth, to hold the lower jaw in a fixed position and prevent it from falling back towards the throat. It is believed that such devices cause discomfort and tend to slip out of position during use, thereby becoming less than fully effective.

Another prior proposal has been to employ a concave head pillow which engages the lower jaw, pushing it forward as gravity pulls the person's head into the pillow. The pillow is believed to be effective only when the user is in a supine position, and only if the user's head maintains a roughly fixed position relative to the pillow's active contact points. The pillow's effectiveness is believed to be reduced if the person is lying on his or her side or stomach, or if the person turns the head relative to the pillow. In addition, the pillow's size makes it inconvenient for travel or storage.

A further prior suggested solution is to place a small pillow around a person's neck, filing up the space between the chin and the throat, thereby employing the pillow's volume to help prevent the lower jaw from falling open or moving posteriorly. The pressure applied by this device is primarily directed upwards toward the top of the skull, with only a portion of the force acting in a forward direction. It is believed that at least some users will find such neck pillows uncomfortable and of limited effectiveness.

Another prior proposal is to use a rigid U-shaped or similar hard fixture under the lower jaw or its rear corners, with a head strap and an across-the-face strap and appliance structure to pull upwardly and forwardly on the fixture. The rigid fixture and appliance structure are believed to be awkward and/or uncomfortable to wear, unduly costly, relatively difficult to use, and of limited effectiveness.

Further prior art proposals suggest a chin strap connecting the area around the chin to the top of the skull. Rather than applying forward pressure to the lower jaw, such straps primarily act to prevent the user's mouth from falling open during sleep. It is believed that such proposals are of only limited effectiveness.

### OBJECTS OF THE INVENTION

It is an object of the present invention to provide an effective sleep disorder relief device and method which eliminates or alleviates the foregoing problems.

It is an object to provide such a device which is compact, relatively simple to use, comfortable to wear, and inexpensive to manufacture.

It is a further object of the invention to provide such a device which is capable of being configured by the user to meet the user's individual needs and preferences for the overall configuration of the device, including the location of strap attachment points, and the amount of pressure exerted by the device.

### SUMMARY OF THE INVENTION

In one embodiment, the device for urging the jaw forwardly includes first and second bands or straps, preferably elastic, the first of which wraps around the head and has a soft chin section which fits under the jaw of the wearer, and the second of which extends across the front of the head of the wearer and is attached to the first band to pull it forwardly and thereby hold the jaw in a forward position.

Preferably, the first and second bands are secured to one another with an attachment which permits adjustment of the relative positions of the bands and the tension forces applied by those bands.

It also is preferable that the chin section of the first strap is made of soft material such as cloth, with an optional coating of friction-enhancing material to enhance gripping contact between the jaw and the chin section.

Preferably, the bands have contact fasteners such as book-and-loop fabric near at least one end to allow adjustment of their lengths.

In one embodiment, the second band includes eye coverings to utilize the device to block sleep-depriving light from the wearer's eyes, and thus further enhance sleep.

The bands preferably comprise elastic bandage material, with or without hook-and-loop contact fastener material at selected ends of the bands to provide tension adjustment means. Alternatively, the bands are made of largely inelastic material, such as cloth, and fasteners to adjust the tension in the bands.

In another embodiment, a first elastic band has a soft chin portion which fits under the lower jaw, and another portion which extends across the front of the wearer's head, and a second elastic band which is attached to the first band at positions on opposite sides of the wearer's face, and extends around the wearer's head.

In the method of use, the device is attached with the soft chin section under and wrapped around the sides of the chin, with an attached band extending around the wearer's head.

The bridge is positioned across the front of the head of the wearer, at a selected location, and is attached and stretched to pull forwardly on the sides of the chin portion, and thus hold the jaw of the wearer in a forward position. This combats snoring and sleep apnea, and thus promotes restful sleep by the wearer and others within earshot of the wearer.

The positions of the bands and the tension forces applied by the bands can be adjusted by shifting the contact points between the first and second loops and the bridge, and/or by the contact attachment devices (e.g., hook-and-loop material patches) which can be used to adjust the length of the straps.

The foregoing and other objects and advantages of the invention will be set forth in or apparent from the following description and drawings.

### IN THE DRAWINGS

FIGS. 1A and 1B are perspective, partially schematic views of one embodiment of the invention;

FIG. 2 is a partially schematic view of the device of FIGS. 1A and 1B, illustrating how the device is attached to the user's head;

FIG. 3 is a view like that of FIG. 2 showing a second embodiment of the invention;

FIG. 4 is a bottom plan view, partially broken away, showing the underside of the chin of the wearer of the device;

FIGS. 5A and 5B are, respectively, side elevation and front elevation views, partially schematic, of the head of a person wearing one embodiment of the invention;

FIGS. 6A and 6B are, respectively, side elevation and front elevation views of a person's head who is wearing an embodiment of the present invention, with an operative part of the embodiment in a location different from that shown in FIGS. 5A and 5B;

FIGS. 7A and 7B are, respectively, side elevation and front elevation views of a person's head who is wearing another embodiment of the invention;

FIGS. 8A and 8B are, respectively, side elevation and front elevation views of a person's head who is wearing yet another embodiment of the invention;

FIG. 9 is a perspective view of still another embodiment of the invention;

FIGS. 10A and 10B are, respectively, side elevation and front elevation views of a head of a person wearing the invention embodiment shown in FIG. 9; and

FIGS. 11, 12 and 13 are, respectively, a perspective view, and two side elevation views of another embodiment of the invention.

### DETAILED DESCRIPTION

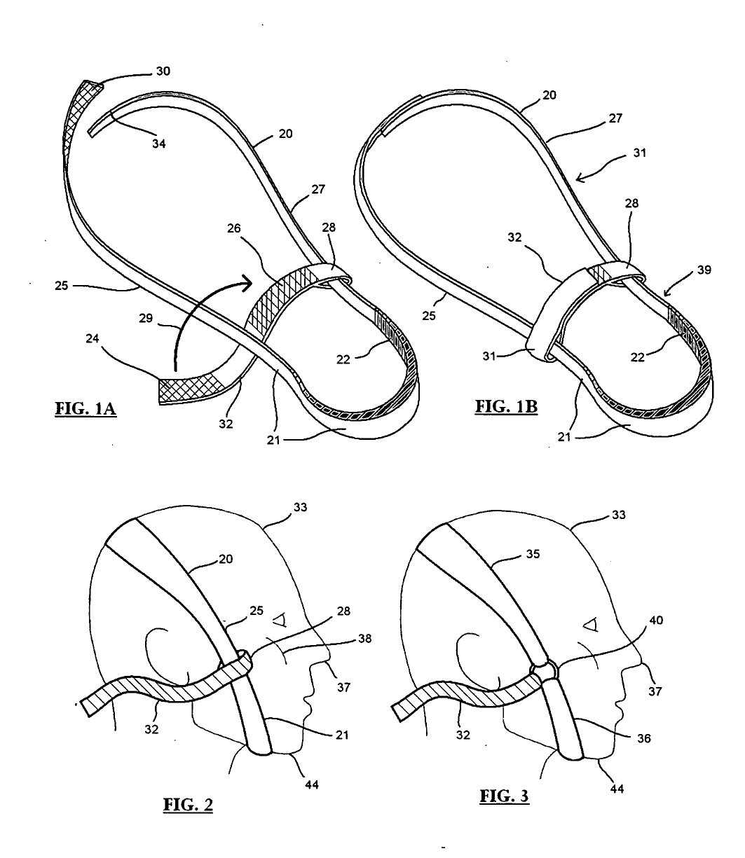

FIGS. 1A, 1B and 2 show one embodiment of a jaw-thrusting device made in accordance with the present invention. The device includes a first strap or band 20 which is long enough to wrap around the head of the wearer and under the chin. The strap preferably is made of elastic material so that it can be tightened to apply pressure to the undersurface of the jaw.

The chin portion 21 which fits underneath the jaw of the wearer preferably is of soft material so as to be comfortable to the wearer even though the strap is applying pressure underneath his or her jaw.

In the embodiment shown in FIGS. 1A, 1B and 2, the strap 20 has two ends, 30 and 34, with hook-and-loop material such as “Velcro” brand material near the ends. The two ends can be adhered together at different longitudinal positions so as to vary the length of strap 20 and the amount of tension it applies to the jaw.

The chin section 21 of the strap has an optional coating 22 of material for increasing the coefficient of friction of the chin section 21 to enhancing the gripping of the jaw.

A second strap 32, called a “frontal” strap or a “bridge,” also preferably of elastic material, has a loop 28 formed at one end, and hook-and-loop material segments 24 and 26 at the other end.

The frontal strap 32 is attached to the sides 25 and 27 of the strap 20 by slipping one end 34 of the strap 20 through the loop 28 of the frontal strap 32, and wrapping the end 24 of the strap 32 around the left portion 25 of the strap 20 by motion in the direction indicated by the arrow 29 until the end 24 makes contact with a desired portion of the patch of hook-and-loop material 26 to secure the left end of the frontal strap to the first strap 20. By adjusting the length of the strap 32, the amount of forward thrust on the wearer's jaw is adjusted.

FIG. 1B shows the finished assembly. Ends 30 and 34 of the strap 20 are attached together, and the frontal strap 32 is attached to the sides 25 and 27 by means of loops 28 and 31 at the ends of the frontal strap.

In accordance with one feature of the invention, the loops 28 and 31 preferably are large enough to allow the material of the strap 20 to slide through the loops when manipulated by hand so as to adjust the position of the loops 28 and 31 along the length of the strap 20 for adjustment purposes. This can be used to adjust the distribution of tension applied by the straps, including the tension of the strap under the jaw.

Now, referring to FIG. 2, the strap 20 is shown wrapped around the head 33 of a wearer, with the chin portion 21 extending underneath the jaw 44 of the wearer.

Although the strap 20 is shown forming an angle of around 45 degrees in the position shown in FIG. 2, it should be understood that the position of the strap 20 can be moved upwardly or downwardly on the wearer's head, depending upon the preference of the wearer, and depending upon how much tension the wearer wishes to apply to the jaw.

The frontal strap 32 is shown in FIG. 2 before being attached to the side 25 of the strap 20. Loop 28 is shown wrapped around the side portion 25 of the strap 20, (this is the reverse of the showing in FIGS. 1A and 1B). The frontal strap 32 is ready to be wrapped over or under the nose 37 of the wearer, and perhaps in contact with the cheekbones 38 of the wearer's face.

Another way of describing the embodiment of FIGS. 1A, 1B and 2, and other embodiments to be described below, is that two elastic bands or straps are attached together to form two loops.

One loop 31 (FIG. 1B) is formed by the upper portions of strap 20 with the strap 32, which is referred to here as a “bridge” strap because it bridges the two sides 25 and 27 together.

The other loop 39 is formed by the combination of the bridge strap 32 with the chin portion 21.

The relative sizes of the loops, and the position of the bridge can be changed to adjust the device for maximum comfort and effectiveness.

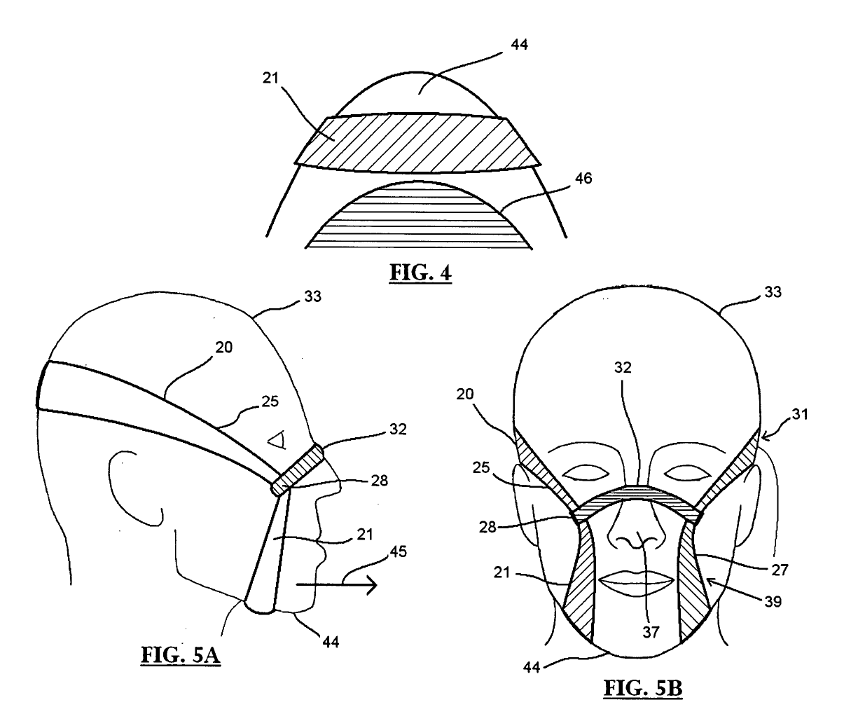

FIG. 3 shows another embodiment of the invention. It is same as the embodiment shown in the previous Figures, except that, instead of a single band 20, two bands 35 and 36 are provided and separately attached to a metal or plastic ring 40. The frontal strap or bridge 32 is attached at one end to the ring 40, and then can be looped over the face and attached to another ring like ring 40 (not shown) on the other side of the wearer's face in the same manner as that used to attach the loose end of the frontal strap 32 in FIG. 2. This embodiment of the invention provides a fixed attachment point for the frontal strap 32, rather than the variable attachment point provided in the embodiments shown in the previous figures of the drawings.

When the embodiment of FIG. 3 is used, preferably, the strap 35 is made with two ends, 30 and 34 (FIG. 1A) with hook-and-loop fasteners to allow adjustments to the length of the strap 35 to vary the amount of tension applied to the chin portion 36.

Still referring to FIG. 3, optionally, the chin portion 21 can have separate open ends with hook-and-loop attachment material to permit separate adjustment of the amount of tension provided in the chin portion 21. By use of the separate adjustment of the lengths of the two loops, tensions can be adjusted as needed and desired without adversely affecting the position of the rings 40 and the bridge member 32. Alternatively, those adjustment devices can be used to move the position of the rings 40.

FIG. 4 shows the bottom of the wearer's jaw at 44, and the neck portion of the wearer at 46. The chin portion 21 of the strap is shown extending underneath the jaw portion.

FIGS. 5A and 5B show the fully assembled device of FIGS. 1A, IB and 2. The frontal strap or bridge 32 is positioned over the bridge of the nose 37 of the wearer. This tends to pull chin portion 21 in a forwardly direction, indicated by arrow 45, to urge the lower jaw forwardly for the purpose of combating sleep disorders.

FIG. 5B shows a partial view of the two loops, 31 and 39, that are formed, one by the combination of the bridge 32 with the upper portion of the strap 20, and the other by the bridge 32, in combination with the lower portion 21 of the strap 20.

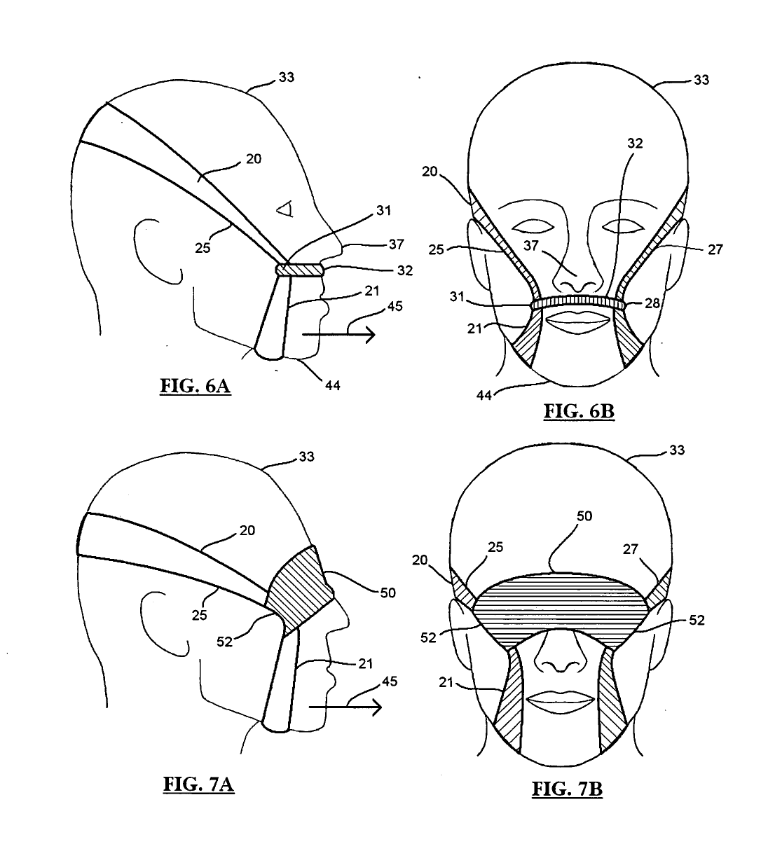

FIGS. 6A and 6B show a variation in the positioning of the frontal strap or bridge 32 to a location underneath the nose 37 of the wearer. This adjustment, which can be adopted, if desired, for the comfort for the wearer, and/or for increased effectiveness in holding the jaw in a forwardly position, can be made easily by simply slipping the band 20 through the loops 31 and 28 at the end of the bridge member 32 and moving the bridge member downwardly and adjusting the length of the bridge 32 to produce the desired amount of forward thrust on the jaw.

As in the previous figures of the drawings, the lower jaw 44 of the user is urged forwardly in the direction of the arrow 45.

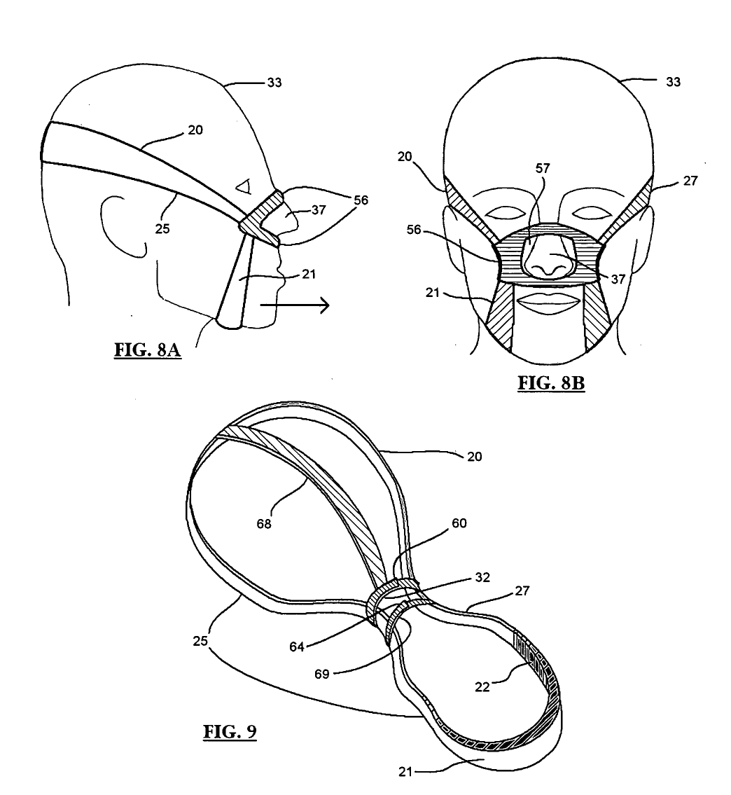

FIGS. 7A and 7B show a modified form of the invention shown in the previous figures of the drawing.

Instead of the strap 32 forming the frontal strap or bridge, a piece of stretchable opaque or semi-opaque material 50 is attached between the opposite sides 25 and 27 of the strap 20 by means of attachments 52 like the attachments 31 and 28. The material 50 is positioned by the wearer so as to comfortably cover the eyes, while simultaneously providing forwardly-directed tension on the chin portion 21 to urge the jaw forwardly in the direction of arrow 45.

Thus, the invention makes dual use of the bridge to provide both forward tension on the chin portion 21, and an eyeshield. The shield will greatly reduce ambient light reaching the eyes, and will prevent the light from disturbing the user, thus further promoting sound sleep.

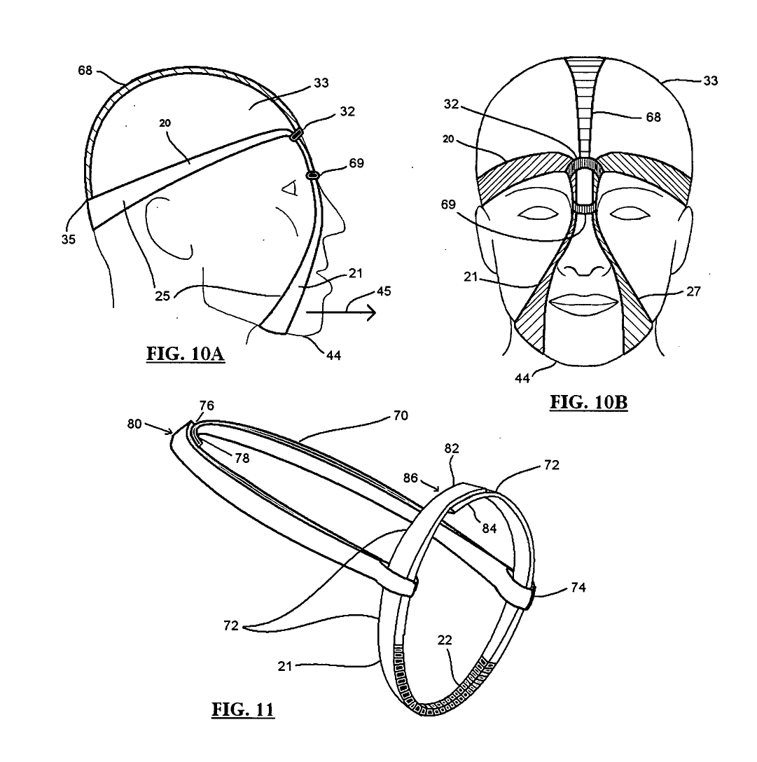

FIGS. 8A and 8B show another embodiment of the invention in which the frontal strap or bridge 32 is replaced by an appliance 56, formed of elastic or other soft material which is secured to the sides 25 and 27 of the strap 20. The appliance 56 has a central opening 57, which is large enough to accommodate the nose 37 of the wearer. The use of the appliance 56 can be more comfortable for some wearers, and also can help hold the device in place despite the user tossing and turning in bed. As with the embodiment shown in FIGS. 7A and 7B, the appliance 56 is attached to the strap 20 so as to be slidable along the length of the strap 20 to adjust the position of the appliance.

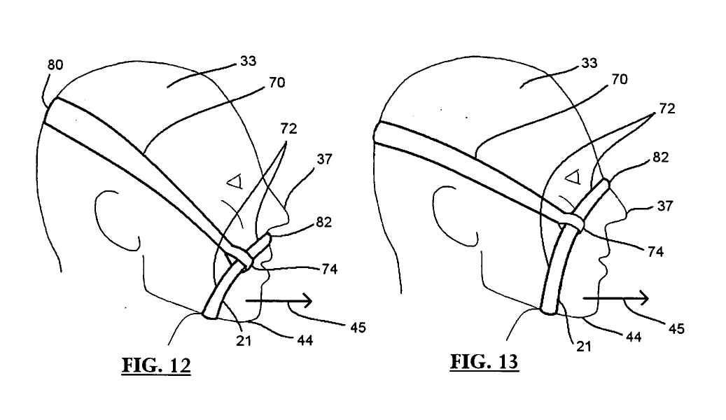

FIG. 9 and FIGS. 10A and 10B show another embodiment of the invention, in which the first strap 20 can be positioned lower on the head 33 of the wearer than in any previous embodiment. The central portions of the band 20 are pulled closely together in an area above the nose 37 on the forehead of the wearer by a relatively short bridge or frontal strap 32 and a second bridge strap 69 parallel to the strap 32. As shown in FIG. 9, the frontal straps or bridges 32 and 69 are fastened into a loop by hook-and-loop attachment means at 60 and 64. Similar attachment means can be used to attach the additional straps 68 at its ends to the straps 32 and 20.

Another strap 68 is attached to the strap 32 at the front of the wearer's head, and at 35 (FIG. 10A) at the rear of the head. The strap 68 extends over the top of the head 33, and tends to hold the straps 32 in place. Also, it is believed that the strap tends to stay in position all night better than in some of the embodiments shown above. As with other embodiments, the various straps can be made adjustable in length by providing Velcro strips near the ends of the strap portions.

FIGS. 11, 12 and 13 show a different embodiment of the invention, in which two straps are provided, one strap 70 forming a loop extending around the back of the head of the wearer, and the other forming loop 72, which extends under the chin 44 and under or above the nose 37 of the wearer. The strap 70 is attached at two points 74 hold the device on the wearer's head. Adjustable attachments are provided at 80 for ends 76 and 78 of band 70, and at 86 for ends 82 and 84 of the strap 72.

FIG. 12 shows the device of FIG. 11 with the loop 72 located under the nose 37 of the wearer, and FIG. 13 shows the loop 72 above the nose 37.

Each of the attachments 74 is by means of a loop such as the loops 31 and 28 shown in FIG. 1 so that the attachment points between the two loops and some of the tensions in the straps can be adjusted by the wearer.

### Materials

As noted above, it is preferred that the straps 20, 32, etc. be made of elastic material such as elastic bandage material, or other stretchable, resilient material. However, inelastic materials such as woven cloth, plastic, or other flexible materials which stretch very little also can be used.

It should be understood that, by the use of the term “elastic” for any strap, it is intended to include straps which have woven cloth or other inelastic sections combined with elastic sections so that the composite strap can be stretched in length and will return to its original condition when released.

It is preferred that the material of the straps does not adhere to the hair of the wearer or the bedclothes of the bed in which the wearer is sleeping. However, the chin portion 21 preferably has a coating 22 on its inside surface of high-coefficient of friction, flexible material such as silicone rubber or silicone gel, (like the gel used in shoe insoles). By thus adding additional gripping capability to the chin section 21, the device better resists the shifting of the device which might occur from the person twisting and turning in bed during a night's sleep, and also resists slippage under the forward pull of the elastic straps.

Alternatively, other materials can be used for the straps. For example, soft, flexible and/or elastic materials such as neoprene, spandex, rubber or similar materials can be used.

If desired, padding can be added to the straps to make them more comfortable. However, elastic bandages already are designed with that purpose in mind and are economical and readily available. Also, although hook-and-loop material attachment devices have been described as preferred, buckles and other clasps can be used in their place, if desired.

If desired, adhesives can be used in the chin portion 21 of the device to assist adherence of the chin portion to the underside of the user's chin.

Although the invention has thus been shown and described with reference to specific embodiments, it should be noted that the invention is in no way limited to the details of the described arrangements but changes and modifications may be made without departing from the scope of the invention which is defined by the appended claims.

1. A device for urging the lower jaw of a person forwardly during sleep to combat sleeping disorders, said device comprising
(a) two flexible straps secured together to form two loops,
(b) one of said loops having a soft chin portion for fitting under the chin,
(c) the other of said loops extending around the head of said person,
(d) said straps forming a bridge across the front of the person's head to pull forwardly on said chin portion.

2. A device as in claim 1 in which the attachment points between said bridge and said loops are movable to adjust said straps, the material of which said straps are made is selected from the group consisting of an elastic material and an in-elastic material.

3. A device as in claim 1 in which said bridge has two branches with a space in between to accommodate the wearer's nose.

4. A device as in claim 1 in which said bridge includes an eye shield of a size sufficient to cover the eyes of the wearer and being made relatively opaque.

5. A device as in claim 1 including a second bridge portion parallel to said first bridge portion, and a further elastic strap joined at one end to one of said bridge portions to fit over the top of the wearer's head to connect to the one of said loops which extends over the wearer's head.

6. A device as in claim 1 in which said chin portion is made of soft material with a frictional engagement-enhancing covering thereon.

7. A device as in claim 1 including a length-adjustment attachment for attaching the ends of at least one of said loops together to make the length of that loop adjustable.

8. A device for forwardly urging the lower jaw of a human being, said device comprising
(a) A first flexible band having a soft chin section for fitting under the lower jaw of said human being,
(b) Said first band being dimensioned for wrapping around the head of the wearer and having opposite side sections and being stretched to pull on said chin section when said chin section is positioned underneath the lower jaw of said wearer, and
(c) A frontal section comprising a second flexible band extending across the front of the head of said wearer and having two ends, each end being secured to one of said opposite sides of said first band to pull forwardly on said opposite sides when said device is worn by said wearer and thereby forwardly urge said lower jaw.

9. A device as in claim 8 in which each of said ends of said second elastic band is secured to one of said opposite sides of said first band by an attachment structure which allows the position of said end along the length of said first band to be adjustable.

10. A device as in claim 8 in which said bands are made of a material selected from the group consisting of elastic material and non-elastic material.

11. A device as in claim 8 in which said first band has a length adjustment device to vary the pressure applied by said first band to said chin section.

12. A device as in claim 8 in which said chin section comprises a material which has a coating of friction-enhancement material to enhance frictional engagement between said lower jaw and said chin section.

13. A device as in claim 11 in which said length adjustment device comprises a pair of ends for said first band, and hook-and-loop attachment material distributed along the length of said first band adjacent each of said ends.

14. A device as in claim 13 in which said second band has a pair of ends, each with a hook-and-loop securing structure for releasably attaching to one of said side sections of said first band.

15. A device as in claim 8 in which each of said first and second bands includes at least one elastic bandage with at least one of said second band and said first band having an attachment device for attaching said first and second bands together at said opposite sides of said first band.

16. A device as in claim 8 in which each of said ends of said second band is wrapped around said first band so as to create light frictional engagement with said first band, said engagement being light enough to permit the points of attachment of said ends to be slid along the length of said first band.

17. A device as in claim 8 in which said frontal section includes an eye shield to cover the eyes of the wearer.

18. A method of combating sleep disorders, said method comprising the steps of
(a) providing a device for forwardly-urging the lower jaw of a person, said device comprising:
(1) two flexible straps secured together to form two loops,
(2) one of said loops having a soft chin portion for fitting under the chin,
(3) the other of said loops extending around the head of said person,
(4) said loops forming a bridge across the front of the person's head to pull forwardly on said chin portion.
(b) wrapping said straps around the wearer's head with said chin portion under said chin, and
(c) positioning said bridge portion across a selected portion of the front of the wearer's head with a tension sufficient to forwardly urge said lower jaw and hold said lower jaw in a forward position during sleep.

19. A method as in claim 18 including the further step of:
(d) setting the tension of the strap forming at least one of said loops at a level effective to minimize shifting of said lower jaw from a forward position during sleep.

20. A method as in claim 18 in which the strap forming at least one of said loops has two ends and the positions of said ends of said band can be shifted by hand, and including shifting said positions to maximize the comfort of the wearer while providing tension sufficient to forwardly urge the jaw of the wearer.
